(19) United States
(12) Patent Application Publication
(10) Pub. No.: US 2009/0054775 A1
(43) Pub. Date: Feb. 26, 2009

(54) ULTRASONIC DIAGNOSTIC APPARATUS AND ULTRASONIC IMAGING METHOD

(76) Inventors: Sei Kato, Tokyo (JP); Takanori Saito, Tokyo (JP)

Correspondence Address: PATRICK W. RASCHE (20459);ARMSTRONG TEASDALE LLP, ONE METROPOLITAN SQUARE, SUITE 2600, ST. LOUIS, MO, 63102-2740, US

(21) Appl. No.: 12/192,914

(22) Filed: Aug. 15, 2008

(30) Foreign Application Priority Data
Aug. 21, 2007 (JP) 2007-214774

(51) Int. Cl. A61B 8/00

(52) U.S. Cl. 600/443; 382/128

(57) ABSTRACT

An ultrasonic diagnostic apparatus includes: a contrast image creating device which transmits each of contrast imaging transmission pulses and creates a contrast image, based on a received signal corresponding contrast imaging transmission pulse; a first reference image creating device which creates a reference image using the received signal corresponding to the contrast imaging transmission pulse; a second reference image creating device which transmits a reference transmission pulse different in frequency from the contrast imaging transmission pulse and creates a reference image, based on a received signal corresponding to the reference transmission pulse; a switching device which selects one of the first reference image creating device and the second reference image creating device and activates the same; and an image display device which displays the contrast image and the reference image side by side or displays the same in superimposed form.

### CROSS REFERENCE TO RELATED APPLICATIONS

This application claims the benefit of Japanese Patent Application No. 2007-214774 filed Aug. 21, 2007, incorporated by reference herein in its entirety.

### BACKGROUND OF THE INVENTION

The subject matter disclosed herein relates to an ultrasonic diagnostic apparatus, and more specifically to an ultrasonic diagnostic apparatus capable of optimizing a balance between a frame rate of a contrast image and the quality of a reference image.

There has been known an ultrasonic diagnostic apparatus which transmits each of contrast imaging transmission pulses and creates a contrast image, based on a received signal corresponding thereto, and further transmits each of reference transmission pulses and creates a reference image, based on a received signal corresponding thereto, and which combines the contrast image and the reference image together and displays the result of combination (refer to, for example, Japanese Unexamined Patent Publication No. 2002-045360 and Japanese Unexamined Patent Publication No. 2003-052698).

In the ultrasonic diagnostic apparatus that transmits the reference transmission pulse and creates the reference image, based on the received signal corresponding thereto, a reference image good in image quality is obtained. A problem, however, arises in that since the frame rate of the contrast image is lowered by the transmission of the reference transmission pulse, the present apparatus fails to adapt to a case where the frame rate of the contrast image is emphasized.

On the other hand, if no reference transmission pulse is transmitted and a reference image is created using a received signal corresponding to each of contrast imaging transmission pulse, then the frame rate of a contrast image is not lowered. A problem, however, arises in that since the reference image is degraded in image quality as compared with the reference image created based on the received signal corresponding to the reference transmission pulse after the transmission of the reference transmission pulse, it fails to adapt to a case where the quality of the reference image is emphasized.

### BRIEF DESCRIPTION OF THE INVENTION

It is desirable that the problems described previously are solved.

In a first aspect, the invention provides an ultrasonic diagnostic apparatus including a contrast image creating device which transmits each of contrast imaging transmission pulses and creates a contrast image, based on a received signal corresponding thereto, a first reference image creating device which creates a reference image using the received signal corresponding to the corresponding contrast imaging transmission pulse, a second reference image creating device which transmits a reference transmission pulse different in frequency from the contrast imaging transmission pulse and creates a reference image, based on a received signal corresponding thereto, a switching device which selects one of the first reference image creating device and the second reference image creating device and activates the same, and an image display device which displays the contrast image and the reference image side by side or displays the same in superimposed form.

In the ultrasonic diagnostic apparatus according to the first aspect, a frame rate of a contrast image is not lowered because no reference transmission pulse is transmitted if the first reference image creating device is selected. Since the reference transmission pulse is sent if the second reference image creating device is selected, a reference image good in quality is obtained. That is, the balance between the frame rate of the contrast image and the quality of the reference image can be optimized as the case may be.

In a second aspect, the invention provides an ultrasonic diagnostic apparatus wherein in the ultrasonic diagnostic apparatus according to the first aspect, the switching device performs the selection according to instructions of an operator.

The ultrasonic diagnostic apparatus according to the second aspect is capable of switching based on an operator's decision whether to select the first reference image creating device or to select the second reference image creating device.

In a third aspect, the invention provides an ultrasonic diagnostic apparatus wherein in the ultrasonic diagnostic apparatus according to the first or second aspect, the switching device performs the selection according to FOV.

When FOV (index indicative of how depth is observed) is deep, there is no allowance for the frame rate of the contrast image. When FOV is shallow, allowance occurs in the frame rate of the contrast image. Thus, in the ultrasonic diagnostic apparatus according to the third aspect, the selection of the first reference image creating device and the second reference image creating device is automatically done according to FOV.

In a fourth aspect, the invention provides an ultrasonic diagnostic apparatus wherein in the ultrasonic diagnostic apparatus according to the third aspect, the switching device selects the first reference image creating device when FOV is greater than or equal to a preset depth and selects the second reference image creating device when FOV is less than the preset depth.

In the ultrasonic diagnostic apparatus according to the fourth aspect, the first reference image creating device is automatically selected when FOV is deeper than, for example, 10 cm and there is no allowance for the frame rate of the contrast image. It is therefore possible to avoid a reduction in the frame rate of the contrast image. Since the second reference image creating device is automatically selected when FOV is shallower than, for example, 10 cm and allowance exists in the frame rate of the contrast image, a reference image good in image quality is obtained.

In a fifth aspect, the invention provides an ultrasonic diagnostic apparatus wherein in the ultrasonic diagnostic apparatus according to the first or second aspect, the switching device performs the selection according to a transmission focus.

Since FOV is also deep in general when the transmission focus is deep, there is no allowance for the frame rate of the contrast image. Since FOV is also shallow generally when the transmission focus is shallow, allowance occurs in the frame rate of the contrast image. Thus, in the ultrasonic diagnostic apparatus according to the fifth aspect, the selection of the first reference image creating device and the second reference image creating device is automatically performed according to the transmission focus.

In a sixth aspect, the invention provides an ultrasonic diagnostic apparatus wherein in the ultrasonic diagnostic apparatus according to the fifth aspect, the switching device selects the first reference image creating device when the transmission focus is greater than or equal to a preset focus depth and selects the second reference image creating device when the transmission focus is less than the preset focus depth.

In the ultrasonic diagnostic apparatus according to the sixth aspect, the first reference image creating device is automatically selected when the transmission focus is deeper than, for example, 9 cm and there is generally no allowance for the frame rate of the contrast image. It is therefore possible to avoid a reduction in the frame rate of the contrast image. Since the second reference image creating device is automatically selected when the transmission focus is shallower than, for example, 9 cm and allowance is generally made to the frame rate of the contrast image, a reference image good in image quality is obtained.

In a seventh aspect, the invention provides an ultrasonic diagnostic apparatus wherein in the ultrasonic diagnostic apparatus according to the first or second aspect, the switching device performs the selection according to a target frame rate.

When the target frame rate (lowest desired frame rate of contrast image) is high, there is no allowance for an actual frame of a contrast image. When the target frame rate is low, allowance occurs in the actual frame rate of the contrast image. Thus, in the ultrasonic diagnostic apparatus according to the seventh aspect, the selection of the first reference image creating device and the second reference image creating device is automatically carried out according to the target frame rate.

In an eighth aspect, the invention provides an ultrasonic diagnostic apparatus wherein in the ultrasonic diagnostic apparatus according to the seventh aspect, the switching device selects the first reference image creating device when the actual frame rate becomes less than the target frame rate upon selection of the second reference image creating device and selects the second reference image creating device when the actual frame rate becomes greater than or equal to the target frame rate even though the second reference image creating device is selected.

In the ultrasonic diagnostic apparatus according to the eighth aspect, the frame rate at the time that the second reference image creating device is selected is calculated. When the calculated actual frame rate becomes less than the target frame rate, the first reference image creating device is selected. When the calculated actual frame rate becomes greater than or equal to the target frame rate, the second reference image creating device is selected. It is thus possible to reliably ensure the target frame rate and obtain a reference image good in image quality when there is an allowance for the frame rate.

In a ninth aspect, the invention provides an ultrasonic diagnostic apparatus wherein in the ultrasonic diagnostic apparatus according to any of the first through eighth aspects, the second reference image creating device changes the frequency of the reference transmission pulse according to FOV.

Although the resolution is increased if the frequency of the reference transmission pulse is set high, attenuation in a subject becomes large. Thus, in the ultrasonic diagnostic apparatus according to the ninth aspect, the frequency of the reference transmission pulse is set low so as to prevent attenuation at a deep portion of FOV from increasing where FOV is deep, thereby suppressing the attenuation. Since attenuation in FOV is low where FOV is shallow, the frequency of the reference transmission pulse is raised to enhance resolution.

In a tenth aspect, the invention provides an ultrasonic diagnostic apparatus wherein in the ultrasonic diagnostic apparatus according to the ninth aspect, the second reference image creating device sets the frequency of the reference transmission pulse to a first frequency when FOV is greater than or equal to the preset depth and sets the frequency of the reference transmission pulse to a second frequency higher than the first frequency when FOV is less than the present depth.

In the ultrasonic diagnostic apparatus according to the tenth aspect, when FOV is larger than or equal to 5 cm, for example, the frequency of the reference transmission pulse is set to, for example, 4 MHz so as to prevent attenuation at a deep portion of FOV from becoming large, thereby suppressing the attenuation. Since attenuation in FOV is low when FOV is less than, for example, 5 cm, the frequency of the reference transmission pulse is set to, for example, 5 MHz thereby to enhance resolution.

In an eleventh aspect, the invention provides an ultrasonic diagnostic apparatus wherein in the ultrasonic diagnostic apparatus according to any of the first through eighth aspect, the second reference image creating device changes a wave run length of the reference transmission pulse according to FOV.

Although the resolution is enhanced if the wave run length (length of wave contained in the whole pulse) of the reference transmission pulse is set short, the ability to attain a deep portion within a subject is degraded. Thus, in the ultrasonic diagnostic apparatus according to the eleventh aspect, the wave run length of the reference transmission pulse is made long so as to avoid a reduction in the ability to attain the deep portion when FOV is deep. Since the influence of the reduction in the ability to attain the deep portion is low when FOV is shallow, the wave run length of the reference transmission pulse is made short to enhance the resolution.

Incidentally, the wave run length is substantially equal to a product of the wavelength and the number of bursts (frequency of a wave contained in the whole pulse).

In a twelfth aspect, the invention provides an ultrasonic diagnostic apparatus wherein in the ultrasonic diagnostic apparatus according to the eleventh aspect, the second reference image creating device sets the wave run length of the reference transmission pulse to a first wave run length when FOV is greater than or equal to the preset depth and sets the wave run length of the reference transmission pulse to a second wave run length shorter than the first wave run length when FOV is less than the preset depth.

In the ultrasonic diagnostic apparatus according to the twelfth aspect, when FOV is greater than or equal to, for example, 5 cm, the wave run length of the reference transmission pulse is set to twice the wavelength so as to avoid a reduction in the ability to attain the deep portion. Since the influence of the reduction in the ability to attain the deep portion is low where FOV is less than 5 cm, for example, the wave run length of the reference transmission pulse is made equal to the wavelength, for example, thereby enhancing the resolution.

In a thirteenth aspect, the invention provides an ultrasonic diagnostic apparatus wherein in the ultrasonic diagnostic apparatus according to any of the first through eighth aspects, the second reference image creating device changes at least one of a transmission f value at the transmission of the reference transmission pulse according to FOV and a reception f value at the reception thereof.

If the transmission f value at the time that the reference transmission pulse is transmitted or the reception f value at the reception of its echo is made large, then the depth of focus becomes large but the resolution is degraded. Thus, in the ultrasonic diagnostic apparatus according to the thirteenth aspect, when FOV is deep, at least one of the transmission f value and the reception f value is made large to extend a focusing range in FOV. When FOV is shallow, at least one of the transmission f value and the reception f value is made small to enhance the resolution.

In a fourteenth aspect, the invention provides an ultrasonic diagnostic apparatus wherein in the ultrasonic diagnostic apparatus according to the thirteenth aspect, the second reference image creating device sets at least one of a transmission f value at the transmission of the reference transmission pulse and a reception f value at the reception thereof to a first transmission f value or a first reception f value when FOV is greater than or equal to a preset depth, and sets at least one of an f value at the transmission of the reference transmission pulse and a reception f value at the reception thereof to a second transmission f value smaller than the first transmission f value or a second reception f value smaller than the first reception f value when FOV is less than the preset depth.

In the ultrasonic diagnostic apparatus according to the fourteenth aspect, when FOV is greater than or equal to 5 cm, for example, the transmission f value at the transmission of the reference transmission pulse or the reception f value at the reception thereof is made large to extend a focusing range. On the other hand, when FOV is less than 5 cm, for example, the transmission f value or the reception f value is made small to enhance the resolution.

In a fifteenth aspect, the invention provides an ultrasonic diagnostic apparatus wherein in the ultrasonic diagnostic apparatus according to any of the first through fourteenth aspects, the frequency of the reference transmission pulse is higher than the frequency of the contrast imaging transmission pulse.

In the ultrasonic diagnostic apparatus according to the fifteenth aspect, the frequency of the reference transmission pulse is set higher than the frequency of a transmission pulse suitable for contrast imaging while contrast sensitivity is being held satisfactorily using the frequency of the transmission pulse, thereby making it possible to enhance the resolution of a reference image.

In a sixteenth aspect, the invention provides an ultrasonic diagnostic apparatus wherein in the ultrasonic diagnostic apparatus according to any of the first through fifteenth aspects, the sound pressure of the reference transmission pulse is lower than that of each contrast imaging transmission pulse.

In the ultrasonic diagnostic apparatus according to the sixteenth aspect, since the sound pressure of the reference transmission pulse is lower than that of the contrast imaging transmission pulse, it can prevent a bad influence exerted on contrast imaging.

According to the embodiments of the ultrasonic diagnostic apparatus described herein, the balance between a frame rate of a contrast image and the quality of a reference image can be optimized as the case may be. That is, when emphasis is put on the frame rate of the contrast image, the frame rate of the contrast image is not lowered because no reference transmission pulse is transmitted if the first reference image creating device is selected. When the quality of the reference image is emphasized, a reference image good in image quality is obtained because no reference transmission pulse is sent if the second reference image creating device is selected.

The embodiments of the ultrasonic diagnostic apparatus described herein facilitate ultrasonic imaging using a new contrast medium or agent.

Further objects and advantages of the present invention will be apparent from the following description of embodiments of the invention as illustrated in the accompanying drawings.

### BRIEF DESCRIPTION OF THE DRAWINGS

FIG. 1 is a block diagram showing a construction of an exemplary ultrasonic diagnostic apparatus.

FIG. 2 is a flow chart showing an exemplary procedure for switching processing using the ultrasonic diagnostic apparatus shown in FIG. 1.

FIG. 3 is a flow chart following FIG. 2.

FIG. 4 is a time chart illustrating transmission sound pressures and transmission timings of contrast imaging transmission pulses where a reference image is created using received signals corresponding to the contrast imaging transmission pulses.

FIG. 5 is a time chart illustrating transmission sound pressures and transmission timings of reference transmission pulses and contrast imaging transmission pulses where the reference transmission pulses are transmitted and a reference image is created based on received signals corresponding to the reference transmission pulses.

FIG. 6 is a flow chart following FIG. 2, according to a second embodiment.

FIG. 7 is a flow chart following FIG. 2, according to a third embodiment.

### DETAILED DESCRIPTION OF THE INVENTION

The invention will be described below in further detail by embodiments illustrated in the drawings. Incidentally, the invention is not limited to or by the embodiments.

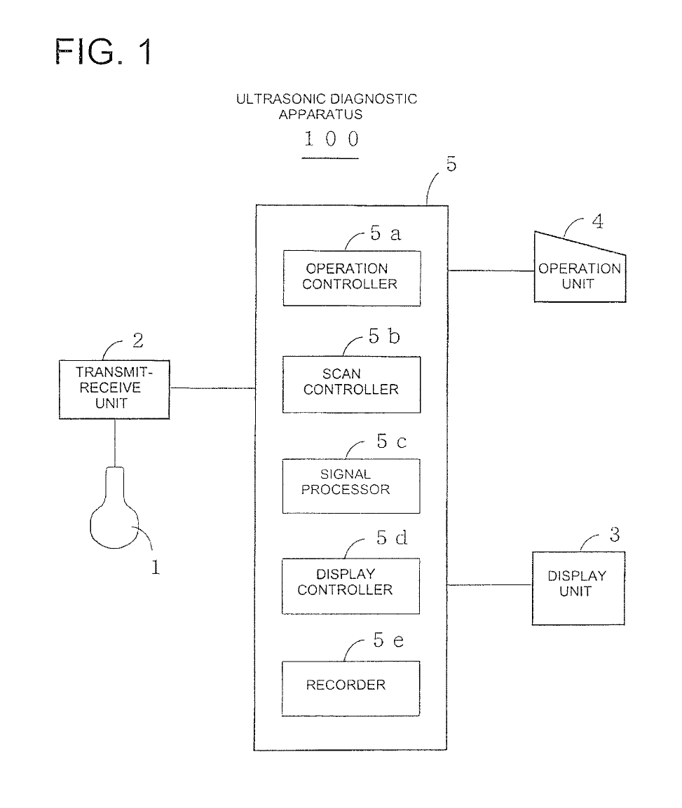

FIG. 1 is a construction explanatory view of an exemplary ultrasonic diagnostic apparatus 100.

The ultrasonic diagnostic apparatus 100 is equipped with an ultrasonic probe 1, a transmit-receive unit 2 which drives the ultrasonic probe 1 to scan within a subject with an ultrasonic beam, a display unit 3 which displays an ultrasonic image and the like thereon, an operation unit 4 for allowing an operator to input instructions and data, and a control unit 5 which controls its entirety.

The control unit 5 includes an operation controller 5a which controls operation relations such as the reception of an input from the operation unit 4, etc., a scan controller 5b which controls scan relations such as a mode (e.g., B mode, CFM or the like) change, a change of scan parameters, etc., a signal processor 5c which performs the processing of each echo signal obtained by the ultrasonic probe 1, the generation of an ultrasonic image and the like, a display controller 5d which controls display relations such as the display of the ultrasonic image and messages on the display unit 3, etc., and a recorder 5e which records the ultrasonic image and the like.

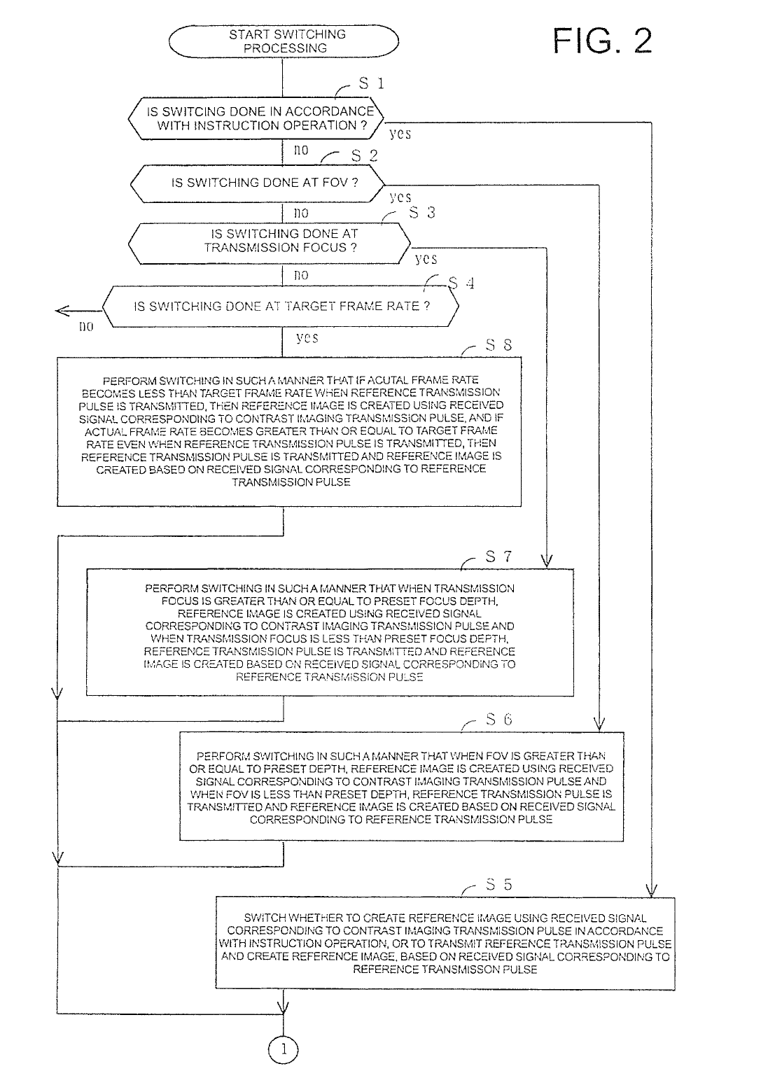

FIG. 2 is a flow chart showing switching or changeover processing done by the ultrasonic diagnostic apparatus 100.

If a switching setting is found not to be taken in accordance with an operator's instruction operation at Step S1, then the switching processing proceeds to Step S2. If the answer is found to be yes, then the switching processing proceeds to Step S5.

If the switching setting is found not to be taken at FOV at Step S2, then the switching processing proceeds to Step S3. If the answer is found to be yes, then the switching processing proceeds to Step S6.

If the switching setting is found not to be taken at a transmission focus at Step S3, then the switching processing proceeds to Step S4. If the answer is found to be yes, then the switching processing proceeds to Step S7.

If the switching setting is found not to be taken at a target frame rate at Step S4, then the switching processing proceeds to another unillustrated processing (error processing, for example). If the answer is found to be yes, then the switching processing proceeds to Step S8.

At Step S5, whether a reference image is created using a received signal corresponding to each of contrast imaging transmission pulses or whether each of reference transmission pulses is transmitted and a reference image is created based on a received signal corresponding thereto, is switched or selected in accordance with instructions inputted by an operator. The switching processing proceeds to Step S11.

If the set FOV is found to be greater than or equal to a depth (10 cm, for example) set in advance at Step S6, then a reference image is created using a received signal corresponding to each of contrast imaging transmission pulses. If the set FOV is less than the depth set in advance, then switching is done in such a manner that each of reference transmission pulses is transmitted and a reference image is created based on a received signal corresponding thereto. The switching processing proceeds to Step S11.

If the set transmission focus is greater than or equal to a focus depth (9 cm, for example) set in advance at Step S7, then a reference image is created using a received signal corresponding to a contrast imaging transmission pulse. If the set transmission focus is less than the focus depth set in advance, switching is then done in such a manner that a reference transmission pulse is transmitted and a reference image is created based on a received signal corresponding to it. The switching processing proceeds to Step S11.

At Step S8, switching is done in such a manner that if an actual frame rate is less than a target frame rate when a reference transmission pulse is transmitted with respect to the set target frame rate, then a reference image is created using a received signal corresponding to a contrast imaging transmission pulse, whereas if the actual frame rate is greater than or equal to the target frame rate even when the reference transmission pulse is sent, then a reference transmission pulse is transmitted and a reference image is created based on a received signal corresponding thereto. The switching processing proceeds to Step S11.

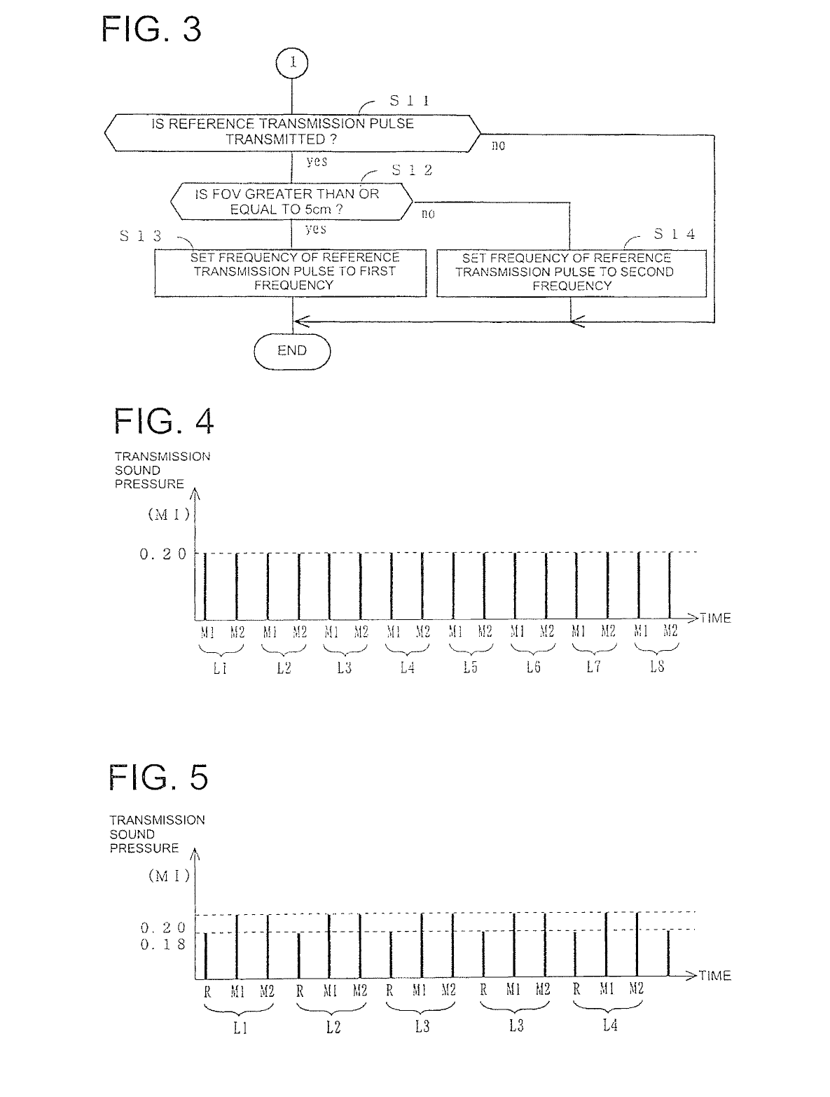

If switching is done at Step S11 of FIG. 3 in such a manner that a reference image is created using a received signal corresponding to a contrast imaging transmission pulse, then the processing is terminated. If switching is done in such a manner that a reference transmission pulse is transmitted, then the switching processing proceeds to Step S12.

If FOV is greater than or equal to a preset depth (5 cm, for example) at Step S12, then the switching processing proceeds to Step S13. If FOV is less than the preset depth, then the switching processing proceeds to Step S14.

At Step S13, the frequency of the reference transmission pulse is assumed to be a first frequency (4 MHz, for example). And the processing is terminated.

At Step S14, the frequency of the reference transmission pulse is assumed to be a second frequency (5 MHz, for example). And the processing is terminated.

FIG. 4 is a time chart illustrating transmission sound pressures and transmission timings of contrast imaging transmission pulses where a reference image is created using a received signal corresponding to each of the contrast imaging transmission pulses.

As in the case where a contrast imaging transmission pulse M1 having a transmission sound pressure 0.2 MI, and a contrast imaging transmission pulse M2 obtained by inverting the phase of a transmission waveform of the contrast imaging transmission pulse M1 are transmitted in a sound ray direction L1 in pairs, next, contrast imaging transmission pulses M1 and M2 each having a transmission sound pressure 0.2 MI are transmitted in a sound ray direction L2 in pairs, . . . , only the contrast imaging transmission pulses are transmitted and no reference transmission pulse is transmitted. Therefore, there is no reduction in frame rate due to the transmission of the reference transmission pulse. Since, however, the frequencies of the contrast imaging transmission pulses M1 and M2 are restricted by a contrast agent, the quality of the reference image created using the received signal corresponding to each of the contrast imaging transmission pulses is deteriorated.

The contrast imaging transmission pulses M1 and M2 are transmitted in pairs to carry out a pulse inversion method. That is, a received signal corresponding to the contrast imaging transmission pulse M1 and a received signal corresponding to the contrast imaging transmission pulse M2 are added together thereby to make it possible to suppress a linear response signal about tissue or the like and efficiently take out a nonlinear response signal from the contrast agent.

The reference image is obtained by applying a receiving filter for extracting a fundamental wave component to either the received signal corresponding to the contrast imaging transmission pulse M1 or the received signal corresponding to the contrast imaging transmission pulse M2. The receiving filter may be configured as a broad band. The reference image is obtained even by performing a subtraction on the received signal corresponding to the contrast imaging transmission pulse M1 and the received signal corresponding to the contrast imaging transmission pulse M2. It is thus possible to enhance sensitivity (S/N).

Setting the frequencies of the contrast imaging transmission pulses M1 and M2 to, for example, 2 MHz enables adaptation to a newly-developed contrast agent, e.g., Sonazoid® (Sonazoid® is a registered trademark of Amersham plc, Buckinghamshire, United Kingdom.

FIG. 5 is a time chart illustrating transmission sound pressures and transmission timings of reference transmission pulses and contrast imaging transmission pulses where the reference transmission pulses are transmitted and a reference image is created based on received signals corresponding to the reference transmission pulses.

As in the case where a reference transmission pulse R having a transmission sound pressure 0.18 MI and contrast imaging transmission pulses M1 and M2 each having a transmission sound pressure 0.2 MI are transmitted in pairs in a sound ray direction L1, next, a reference transmission pulse R having a transmission sound pressure 0.18 MI and contrast imaging transmission pulses M1 and M2 each having a transmission sound pressure 0.2 MI are transmitted in a sound ray direction L2 in pairs, . . . , the reference transmission pulses and the contrast imaging transmission pulses are transmitted. Since the frequency of the reference transmission pulse R is not restricted by a contrast agent, the quality of a reference image created based on the received signal corresponding to each reference transmission pulse R is satisfactory. However, a frame rate is degraded due to the transmission of the reference transmission pulse R.

The reference image is obtained by applying a receiving filter for extracting a fundamental wave component to the received signal corresponding to each reference transmission pulse R. The receiving filter may be configured as a broad band.

If the frequency of the reference transmission pulse R is set to a frequency (3 MHz or higher, for example) higher than the frequencies of the contrast imaging transmission pulses M1 and M2, then the resolution of the reference image can be enhanced.

A method for displaying a contrast image and a reference image has the following variations. In one embodiment, only the contrast image is displayed. In an alternative embodiment, only the reference image is displayed. In another alternative embodiment, the contrast image and the reference image are displayed side by side. In a third alternative embodiment, an image obtained by superimposing the contrast image and the reference image on each other is displayed. In a fourth alternative embodiment, the image obtained by superimposing the contrast image and the reference image on each other, and the reference image are displayed side by side. In a fifth alternative embodiment, the image obtained by superimposing the contrast image and the reference image on each other, and the contrast image are displayed side by side. In a sixth alternative embodiment, the image obtained by superimposing the contrast image and the reference image on each other, the reference image, and the contrast image are displayed side by side.

The pixel values of the image obtained by superimposing the contrast image and the reference image on each other are produced by using an LUT (Look Up Table) which outputs an RGB value with a brightness value of each contrast image and a brightness value of each reference image as inputs.

If, for example, an LUT is defined so as to output an R value proportional to the brightness value of the contrast image, output a B value proportional to the brightness value of the reference image and output a G value=approximately equal to 0, then each pixel at which the brightness value of the contrast image is low and the brightness value of the reference image is high, is displayed blue, each pixel at which the brightness value of the contrast image is high and the brightness value of the reference image is low, is displayed red, each pixel at which the brightness value of the contrast image and the brightness value of the reference image are also high, is displayed purple, and each pixel at which the brightness value of the contrast image and the brightness value of the reference image are also low, is displayed black.

According to the ultrasonic diagnostic apparatus 100 according to the embodiment 1, the following effects are obtained.

(1) When the frame rate of the contrast image is emphasized, the frame rate is not reduced because no reference transmission pulse is transmitted if an operator gives instructions in such a manner that the reference image is created using the received signal corresponding to the contrast imaging transmission pulse (the first reference image creating device). When emphasis is placed on the quality of the reference image, the reference image good in image quality is obtained because the reference transmission pulse is transmitted if the operator gives instructions in such a manner that the reference transmission pulse is transmitted and the reference image is created based on the received signal corresponding thereto (the second reference image creating device).

(2) When there is no allowance for the frame rate, switching can be done automatically in such a manner that the reference image is created using the received signal corresponding to each contrast imaging transmission pulse (the first reference image creating device). When there is an allowance for the frame rate, switching can be done automatically in such a manner that the reference transmission pulse is transmitted and the reference image is created based on the received signal corresponding thereto (the second reference image creating device).

(3) Since the frequency of the reference transmission pulse is automatically changed over according to FOV where the reference transmission pulse is transmitted, the resolution and attenuation can be balanced.

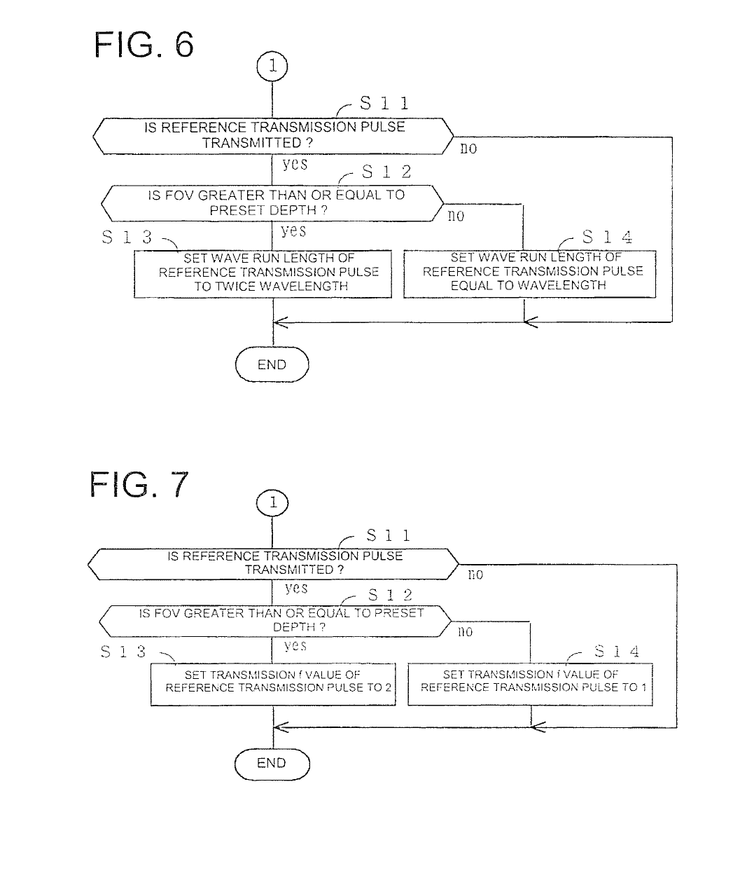

In an alternative embodiment, the processing steps shown in FIG. 6 may be used in place of FIG. 3.

At Step S11 in FIG. 6, the processing is terminated if switching is done in such a manner that a reference image is created using a received signal corresponding to each contrast imaging transmission pulse. If switching is done in such a manner that each reference transmission pulse is transmitted, then the processing proceeds to Step S12.

If FOV is greater than or equal to a preset depth (5 cm, for example) at Step S12, then the processing proceeds to Step S13. If FOV is less than the preset depth, then the processing proceeds to Step S14.

At Step S13, the wave run length of each reference transmission pulse is set to twice the wavelength (the number of bursts is assumed to be 2). The processing is terminated,

At Step S14, the wave run length of each reference transmission pulse is set equal to the wavelength (the number of bursts is assumed to be 1). The processing is terminated.

Since the wave run length is automatically varied according to FOV where the reference transmission pulse is transmitted in the embodiment 2, the image quality and the frame rate can be balanced.

In another alternative embodiment, the processing steps shown in FIG. 7 may be used in place of FIG. 3.

At Step S11 in FIG. 7, the processing is ended if switching is done in such a manner that a reference image is created using a received signal corresponding to each contrast imaging transmission pulse. If switching is done in such a manner that each reference transmission pulse is transmitted, then the processing proceeds to Step S12.

If FOV is greater than or equal to a preset depth (5 cm, for example) at Step S12, then the processing proceeds to Step S13. If FOV is less than the preset depth, then the processing proceeds to Step S14.

At Step S13, a transmission f value of the reference transmission pulse is set to 2. The processing is terminated.

At Step S14, the transmission f value of the reference transmission pulse is set to 1. The processing is terminated.

Since the f value is automatically changed according to FOV where the reference transmission pulse is transmitted in the embodiment 3, the resolution and blurring can be balanced.

Many widely different embodiments of the invention may be configured without departing from the spirit and the scope of the present invention. It should be understood that the present invention is not limited to the specific embodiments described in the specification, except as defined in the appended claims.

1. An ultrasonic diagnostic imaging apparatus comprising:
a contrast image creating device configured to transmit a contrast imaging transmission pulse and to create a contrast, image based on a received signal corresponding to the contrast imaging transmission pulse;
a first reference image creating device configured to create a reference image using the received signal corresponding to the contrast imaging transmission pulse;
a second reference image creating device configured to transmit a reference transmission pulse different in frequency from the contrast imaging transmission pulse and to create a reference, image based on a received signal corresponding to the reference transmission pulse;
a switching device configured to select one of said first reference image creating device and said second reference image creating device and to activate the selected one of said first reference image creating device and said second reference image creating device; and
an image display device configured to display the contrast image and the reference image in one of a side by side form and a superimposed form.

2. The ultrasonic diagnostic apparatus according to claim 1, wherein said switching device is configured to perform the selection according to instructions of an operator.

3. The ultrasonic diagnostic apparatus according to claim 1, wherein said switching device is configured to perform the selection according to an FOV.

4. The ultrasonic diagnostic apparatus according to claim 2, wherein said switching device is configured to perform the selection according to an FOV.

5. The ultrasonic diagnostic apparatus according to claim 3, wherein said switching device is configured to select said first reference image creating device when the FOV is greater than or equal to a preset depth and to select said second reference image creating device when the FOV is less than the preset depth.

6. The ultrasonic diagnostic apparatus according to claim 1, wherein said switching device is configured to perform the selection according to a transmission focus.

7. The ultrasonic diagnostic apparatus according to claim 2, wherein said switching device is configured to perform the selection according to a transmission focus.

8. The ultrasonic diagnostic apparatus according to claim 6, wherein said switching device is configured to select said first reference image creating device when the transmission focus is greater than or equal to a preset focus depth and to select said second reference image creating device when the transmission focus is less than the preset focus depth.

9. The ultrasonic diagnostic apparatus according to claim 1, wherein said switching device is configured to perform the selection according to a target frame rate.

10. The ultrasonic diagnostic apparatus according to claim 2, wherein said switching device is configured to perform the selection according to a target frame rate.

11. The ultrasonic diagnostic apparatus according to claim 9, wherein said switching device is configured to select said first reference image creating device when an actual frame rate becomes less than the target frame rate upon selection of said second reference image creating device and is configured to select said second reference image creating device when the actual frame rate becomes greater than or equal to the target frame rate when said second reference image creating device is selected.

12. The ultrasonic diagnostic apparatus according to claim 1, wherein said second reference image creating device is configured to change the frequency of the reference transmission pulse according to an FOV.

13. The ultrasonic diagnostic apparatus according to claim 12, wherein said second reference image creating device is configured to set the frequency of the reference transmission pulse to a first frequency when the FOV is greater than or equal to a preset depth and is configured to set the frequency of the reference transmission pulse to a second frequency higher than the first frequency when the FOV is less than the preset depth.

14. The ultrasonic diagnostic apparatus according to claim 1, wherein said second reference image creating device is configured to change a wave run length of the reference transmission pulse according to an FOV.

15. The ultrasonic diagnostic apparatus according to claim 14, wherein said second reference image creating device is configured to set the wave length run of the reference transmission pulse to a first wave length run when the FOV is greater than or equal to a preset depth and is configured to set the wave length run of the reference transmission pulse to a second wave length run higher than the first wave length run when the FOV is less than the preset depth.

16. The ultrasonic diagnostic apparatus according to claim 1, wherein said second reference image creating device is configured to change at least one of a transmission f value at the transmission of the reference transmission pulse according to an FOV and a reception f value at the reception of the reference transmission pulse.

17. The ultrasonic diagnostic apparatus according to claim 16, wherein said second reference image creating device is configured to set at least one of a transmission f value at the transmission of the reference transmission pulse and a reception f value at the reception of the reference transmission pulse to a first transmission f value and a first reception f value when the FOV is greater than or equal to a preset depth, and is configured to set at least one of a transmission f value at the transmission of the reference transmission pulse and a reception f value at the reception of the reference transmission pulse to a second transmission f value smaller than the first transmission f value and a second reception f value smaller than the first reception f value when the FOV is less than the preset depth.

18. The ultrasonic diagnostic apparatus according to claim 1, wherein the frequency of the reference transmission pulse is higher than the frequency of the contrast imaging transmission pulse.

19. The ultrasonic diagnostic apparatus according to claim 1, wherein a sound pressure of the reference transmission pulse is lower than a sound pressure of the contrast imaging pulse.

20. An ultrasonic image method comprising the:
transmitting a contrast imaging transmission pulse;
creating a contrast, image based on a received signal corresponding the to contrast imaging transmission pulse;
creating a first reference image using the received signal corresponding to the contrast imaging transmission pulse;
transmitting a reference transmission pulse different in frequency from the contrast imaging transmission pulse;
creating a second reference, image based on a received signal corresponding to the reference transmission pulse;
selecting one of the first reference image and the second reference image; and
displaying the contrast image and the selected reference image in one of a side by side form and in a superimposed form.
